(19) United States
(12) Patent Application Publication
(10) Pub. No.: US 2023/0371829 A1
(43) Pub. Date: Nov. 23, 2023

(54) BLOOD PRESSURE MEASUREMENT DEVICE WITH REMOVABLE CUFF AND METHODS THEREFOR

(71) Applicant: QARDIO, INC., San Francisco, CA (US)

(72) Inventor: Rosario IANNELLA, Amsterdam (NL)

(73) Assignee: QARDIO, INC., San Francisco, CA (US)

(21) Appl. No.: 18/319,545

(22) Filed: May 18, 2023

Related U.S. Application Data
(60) Provisional application No. 63/365,060, filed on May 20, 2022.

(51) Int. Cl. A61B 5/022; A61B 5/00

(52) U.S. Cl. CPC A61B 5/022; A61B 5/0004; A61B 5/742

(57) ABSTRACT

A portable blood pressure measurement device with a removeable/replaceable cuff is disclosed. The device can be capable of wirelessly interacting with a secondary device, such as a cell phone, to measure blood pressure of a patient and configurable to fold into a low profile storage configuration. The removable cuff is sized and configured for wrapping around the limb of a subject the cuff further having an inflatable bladder formed integrally therein; one or more tubes positioned within the cuff in communication with the inflatable bladder; an exhaust valve; a housing connected to the cuff wherein the housing houses one or more of a controller, a memory, a pump, a power supply, and a motor. A transmitter can be provided for wirelessly transmitting blood pressure data to a second device. Operation of the device is furthermore changeable in response to a detected cuff size.

### CROSS-REFERENCE

This application claims the benefit of U.S. Provisional Application No. 63/365,060, filed May 20, 2022, entitled BLOOD PRESSURE DEVICE WITH REMOVABLE CUFF AND METHODS THEREFOR which application is incorporated herein in its entirety by reference.

### BACKGROUND

Regular measurement of blood pressure is an important part of antihypertensive or hypotensive therapy, as well as the control of certain side-effects caused by certain therapies for various kinds of applications. According to the guidelines for antihypertensive therapies of the World Health Organization (“WHO”), the degrees of hypertension are classified in accordance with blood pressure values measured every 5 mmHg, and therapeutic methods suitable for the individual degrees are recommended. Therefore, whether appropriate therapies can be performed depends on the measured blood pressure values. Also, as the number of aging persons in the population increases, demands for high accuracy and high reliability of blood pressure measurement are on the rise in order to prevent circulatory organ diseases and metabolic syndrome which is impacted by hypertension.

Conventionally, a non-invasive sphygmomanometer for measuring the blood pressure uses a cuff wound around an arm of the patient, gradually changing the cuff pressure from a pressure higher than the systolic blood pressure (also called a maximum blood pressure) to a pressure lower than the diastolic blood pressure (also called a minimum blood pressure). A microphone method that measures the blood pressure by detecting the Korotkoff sounds as in the auscultatory method, achieved by listening, such as through a stethoscope, and the oscillometric method that measures the blood pressure by detecting the change in pulse wave superposed on the internal pressure of an internal air bladder of a cuff are used.

Currently, more than 100 million people in the United States suffer from high blood pressure, also referred to as hypertension. Internationally, about 1 in 5 people suffer from high blood pressure. Blood pressure has been called the silent killer because patients often do not detect symptoms while damage to the heart, brain and kidneys occurs. Controlling blood pressure can be achieved by a variety of medications, such as ACE inhibitors, alpha blockers, AR2 blockers, beta blockers, Calcium blockers, diuretics, and vasodilators. Additionally, a variety of life style changes can be employed to control blood pressure.

Patients diagnosed with high blood pressure are advised to engage in a regime of home monitoring—typically once or twice a day—in order to quickly identify changes in blood pressure control. A number of devices are available for home blood pressure measurement. Digital blood pressure devices are considered easy to use: they automatically calculate the pulse and display the systolic and diastolic pressures. However, a simple upper arm blood pressure cuff with a stethoscope is often considered by doctors the most reliable way to measure blood pressure.

Unfortunately, portable blood pressure devices do not easily accommodate patients of difference sizes. What is needed is a convenient way for patients to test blood pressure that allows users to change the size of the cuff and allows the device to detect when the cuff size has changed.

### SUMMARY

Disclosed is a blood pressure measurement device that allows users to change the size of the cuff associated with the device and allows the device to detect the size of the cuff that is attached or when the cuff size has changed. The blood pressure measurement device has an interface between the measurement device and the blood pressure cuff that detects the presence and size of a blood pressure cuff and then adjusts the operation of the blood pressure measurement device in response to the size of the blood pressure cuff. The blood pressure measurement devices can be table-top monitors or portable monitors. The cuff is secured around the user's arm via a cuff securement mechanism.

The disclosed blood pressure measurement devices comprise: a cuff for wrapping around a limb of a subject the cuff further having an inflatable bladder formed integrally therein, wherein the cuff is substantially rectangular in shape having a first surface, a second surface, a first end, a second end, and two sides, one or more tubes positioned within the cuff in communication with the inflatable bladder, an exhaust valve, and a housing mating base secured to a surface of the cuff with a vertical base surface extending from a planar base surface; a housing for the blood pressure measurement device removably securable to the housing mating base of the cuff wherein the housing includes a dock and a button, and houses one or more of a controller, a memory, a pump, a power supply, and a motor wherein the vertical base surface of the housing mating base interfaces with the dock and button of the housing and further wherein the interface between the dock and the button of the housing and vertical base surface is operable to identify a presence and size of the cuff. The blood pressure measurement devices are also capable of blood pressure monitoring.

Methods of measuring and/or monitoring blood pressure using the blood pressure measurement devices comprises: selecting a cuff; securing the cuff to a housing if the blood pressure measurement device; selecting an algorithm for operating the blood pressure measurement device based on a size of the selected cuff; wrapping the cuff around a limb of a subject the cuff having an inflatable bladder formed integrally therein, wherein the cuff is substantially rectangular in shape having a first surface, a second surface, a first end, a second end, and two sides, and a securement mechanism, one or more tubes positioned within the cuff in communication with the inflatable bladder, an exhaust valve; a housing comprising one or more of a controller, a memory, a pump, a power supply, and a motor; and delivering an output of the blood pressure measurement device.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
U.S. Pat. No. 4,776,344 A issued Oct. 11, 1988 by Shirasaki et al.;
U.S. Pat. No. 4,911,169 A issued Mar. 27, 1990 by Ferrari;
U.S. Pat. No. 5,025,791 A issued Jun. 25, 1991 by Niwa;
U.S. Pat. No. 5,595,180 A issued Jan. 21, 1997 by Ogura et al.;
U.S. Pat. No. 5,687,732 A issued Nov. 18, 1997 by Inagaki et al.;
U.S. Pat. No. 5,807,266 A issued Sep. 15, 1998 by Itonaga et al.;
U.S. Pat. No. 5,885,220 A issued Mar. 23, 1999 by Schaer;
U.S. Pat. No. 6,251,080 B1 issued Jun. 26, 2001 by Henkin et al.;
U.S. Pat. No. 6,272,365 B1 issued Aug. 7, 2001 by Ronkainen et al.;
U.S. Pat. No. 7,122,009 B2 issued Oct. 17, 2006 by Fumuro et al.;
U.S. Pat. No. 7,794,405 B2 issued Sep. 14, 2010 by Karo et al.; and
U.S. Pat. No. 8,147,417 B2 issued Apr. 3, 2012 by Gavriely.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-E are views of a blood pressure measurement device with a measurement device body configured to house electronics required to measure blood pressure;

FIGS. 2A-B illustrate a blood pressure measurement device with the measurement device body separated from the cuff and an exploded view of the device;

FIGS. 3A-D illustrate a removable portion of the measurement device body from different perspectives;

FIG. 4 illustrates a blood pressure cuff with a measurement device body base separated from the measurement device body; and

FIG. 5 illustrates another view of a blood pressure cuff with a measurement device body base separated from the measurement device body.

### DETAILED DESCRIPTION

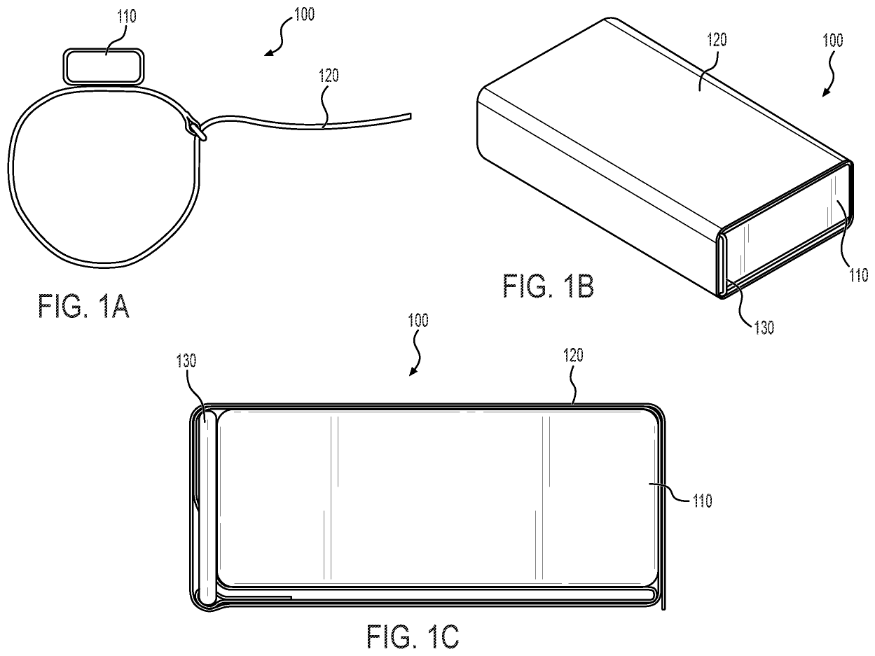

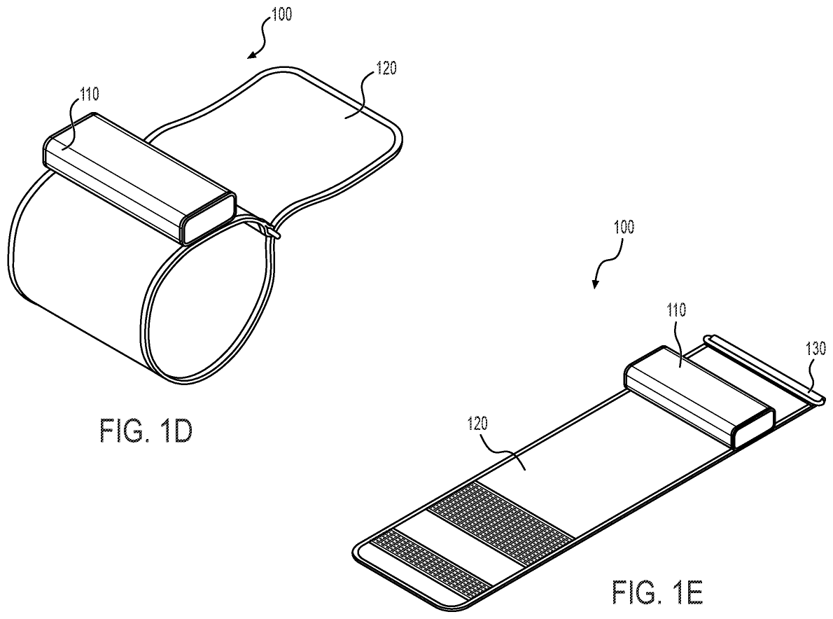

FIGS. 1A-E are views of a blood pressure measurement device 100 with a measurement device body 110 configured to house electronics required to measure blood pressure. The blood pressure measurement device is portable. The blood pressure measurement device 100 includes a cuff member 120 comprising, for example, a compression air bladder, a sub air bladder, and a pulse wave detection air bladder, pressure controller for pressurizing or depressurizing each air bladder, a pressure sensor which is capable of sensing an internal pressure of each air bladder, pulse wave signal extractor for extracting time-series data of a pulse wave signal superposed on a cuff internal pressure. The cuff member 120 is rectangular or substantially rectangular in two dimensions. For example, the rectangular shape in two dimensions can have corners formed at 90 degrees or curved corners. A securement mechanism 130 is also provided. The securement mechanism 130 is an elongated, rectangular shaped ring structure. The cuff member 120 engages the securement mechanism 130 and an end of the cuff member passes through the aperture of the securement mechanism before wrapping around the measurement device body 110 when the device is in a stored/portable configuration.

When the blood pressure measurement device 100 is deployed for use, the pressure controller in the measurement device body 110 pressurizes or depressurizes each air bladder of the cuff member 120, a blood pressure value deriver capable of deriving a systolic blood pressure value and/or a diastolic blood pressure value based on a change in a feature amount of the pulse wave signal and a cuff internal pressure at a point of time of the change. Any wires, tubes or interface elements between the measurement device body 110 and the cuff member 110 are positioned so that the wires, tubes and interface elements are not visible to the user. Other device configurations can be used, such as a table-top monitor, without departing from the scope of the disclosure.

The measurement device body 110 has a height, width and length. The overall shape of the measurement device body 110 can be rectangular with rounded edges along the longest edges as illustrated. A touch location can be provided on the surface for controlling the on-off of the blood pressure measurement device. The touch location can be a flexible indent on the surface on the measurement device body 110 that provides tactile recognition for the user or a button. A light can be associated with the touch location so that the user can detect the on-off control location by the presence of the light. In at least some configurations, no on-off button or touch location is provided. Where there is no on-off button, the blood pressure measurement device is activated when the blood pressure measurement device detects deployment of the blood pressure measurement device, e.g. when the cuff transitions from a storage configuration (wrapped around the measurement device body 110) to a deployed configuration (no longer wrapped around the measurement device body 110). Additionally, the measurement device body 110 can be displayless, can provide for a display or the ability to, for example, provide a visible a blood pressure measurement on the surface of the measurement device body 110 or housing. When the blood pressure measurement device is displayless, the blood pressure measurement can be wirelessly provided to a secondary device with a display in proximity to the blood pressure measurement device, such as a smart phone or tablet. In some configurations, the blood pressure measurement displayed on a surface of the blood pressure measurement device can also be provided wirelessly to a secondary device in proximity to the blood pressure measurement device such that the measurements is available in two locations.

In use, the portable blood pressure measurement device can be used on demand or worn for an extended period of time. When the blood pressure measurement device is worn for an extended period of time, the blood pressure measurement device can be operated on an automatic or semi-automatic basis. For example, the blood pressure measurement device could be set-up to automatically measure blood pressure at set time intervals (e.g., hourly). Alternatively, the blood pressure measurement device could be set-up to measure blood pressure on demand when activated by a user. Additionally, the user could receive a prompt to activate a measurement session.

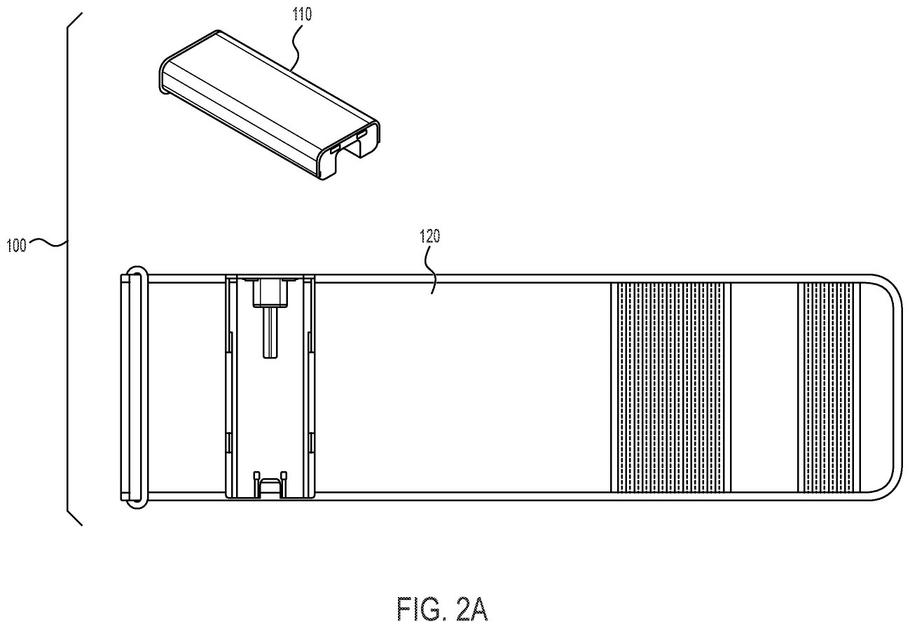

FIG. 2A illustrate a blood pressure measurement device 100 with the measurement device body 110 separated from the cuff member 120. With the measurement device body 110 separable from the cuff member 120, the user can change the size of the cuff that is used with the measurement device body 110. The cuff member 110 has a length and a width. The cuff member 120 incorporates a base 224 which is a platform member that is secured to the cuff member and engages the removable measurement device body 110.

Turning to FIG. 2B, an exploded view of the blood pressure measurement device 100 is illustrated. The measurement device body 110 has a pair of end caps 210, 210′ which engages a cover 212. A plurality of screws 214 is provided to secure a PCB 216 to a dock 220. A pump 240 is further exploded to reveal the use of foam 236, a battery 238, a first and second wire 244, insulation 262, double sided tape 242, 242′, a tube 246, a tee-casing 248, a three-way casing 250, a valve tube 254, a quick-release valve 256, and a plug cover 258. The pump 240 fits within the cover 212, and is positioned on the dock 220. The dock 220 has one or more buttons 222 or control interface surfaces. A base 224 is provided which engages the cuff member 120. A nozzle casing 228 is provided which engages the sleeve tube 230 and the two-way casing 232. Screws 234 secure the nozzle casing to the base 224. The base is a housing mating base that can include a raised structure sized to receive a recess on an exterior surface of the housing.

The cuff member 120 has an attachment mechanism that engages the base 224.

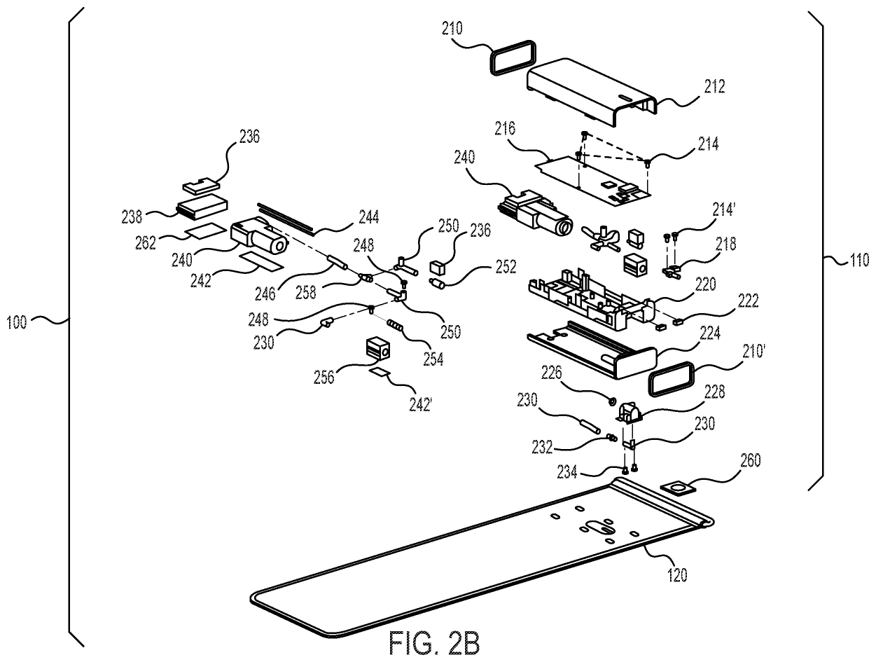

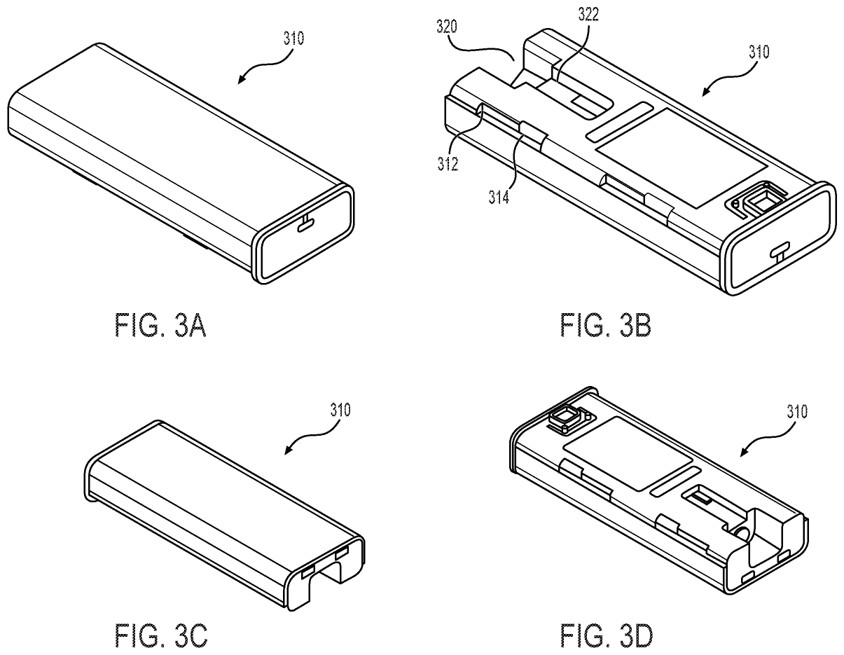

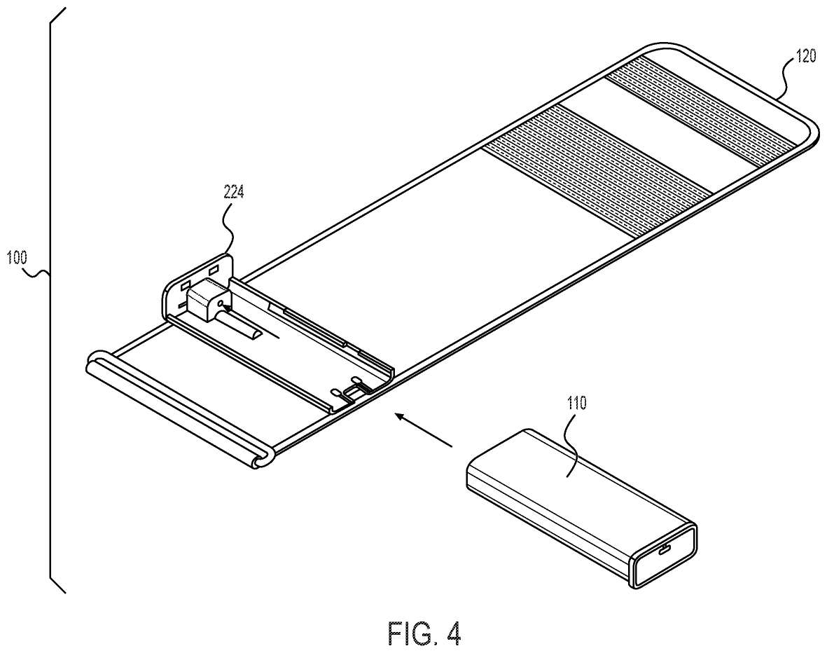

FIG. 3A illustrate a removable body 310 of the measurement device body shown in FIG. 1 from a top first side perspective view. The top first side perspective view illustrates an elongated rectangular shape sized to include electronics, display, speaker, etc. for the blood pressure measurement device. FIG. 3B illustrate the removable body 310 from a bottom first side perspective view. From this view, a side channel 312 and detent 314 is visible along a bottom edge of the removable body 310. More than one channel and detent can be provided on a side, and/or provided on both sides of the removable body. A recess 320 with a channel 322 is provided which is operable to slidably engage the removable body base (shown in FIG. 4). FIG. 3C illustrate a removable portion of the measurement device body 110 from a top second side perspective view. FIG. 3D illustrate a removable portion of the measurement device body 110 from a bottom second side perspective view. When the cuff engages the removable body, the device can either determine the cuff size based on the interface between the cuff and the removable body or via a user interface. In some configurations, the cuff size can be detected and then confirmed via a user interface—either on the device or as part of an app on an electronic device, such as a phone.

FIG. 4 illustrates a blood pressure cuff with a measurement device body base separated from the measurement device body from a top first side perspective view. The measurement device body 110 shown in FIG. 3 slides onto the device body base 224 which is secured to the cuff 120. The device body base 224 has a raised surface 410 that is sized and configured to mate with a portion of the measurement device body. When the one or more buttons 222 on the dock 220 engage a mating interface on the interior surface of the base 224, the presence and size of the blood pressure cuff is identified and provided to allow adjust the operation of the device based on the presence and size of the blood pressure cuff. The change in operation results from a change in the algorithm that takes into account the change of the bladder size resulting from the use of a cuff of a different size.

The one or more buttons 222 are activated differently depending on the cuff that is connected. For example, one or both buttons 222 are sized or constructed to identify the different cuff sizes. The base 224 has one or more pronounced parts that engage the one or more buttons 222 to trigger activation of the cuff and identify the size of the cuff attached, e.g., by applying different amounts of pressure when the base 224 pronounced parts engage the one or more buttons 222.

The cuff size can then be transmitted to an app on a secondary device, such as a mobile device in communication with the blood pressure measurement device. The cuff size can be automatically detected and/or the user can confirm the size of the cuff.

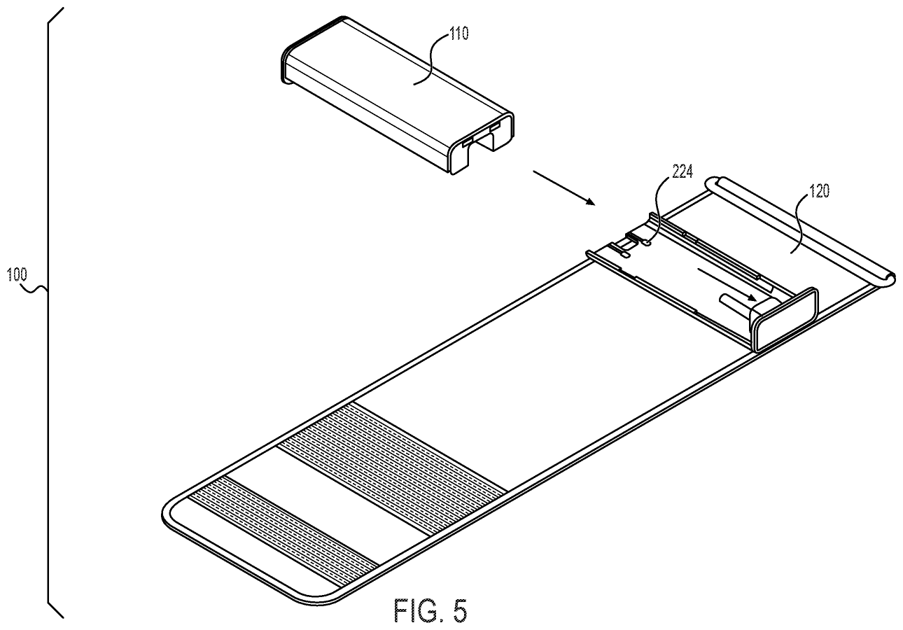

FIG. 5 illustrates another view of a blood pressure cuff with a measurement device body base separated from the measurement device body. When the measurement device body is secured to the cuff, the measurement device body and cuff remains secured together as a single device. The two buttons can allow the device to be switched on and off the other button is used to determine the type of cuff secured to the measurement device body. The operation of the device can change as a result of a change in the cuff size or in response to a user pressing a button. For example, a different cuff size could use a different algorithm to improve accuracy of the blood pressure measurement.

In use, to pressurize and depressurize the cuff member 120 which is adapted to be secured around (pulled tight), for example, a patient's arm, a pump is provided which has a connector which enables pressurizing/depressurizing connected to a compression air bladder of the cuff main body via a second tube and tube, to a pulse wave detection air bladder of the cuff main body via a first tube and fluid resistor, and to the sub air bladder of the cuff main body via a third tube and opening/closing valve. Also, a pressure sensor as a cuff pressure detector for obtaining a cuff pressure signal from the change in pressure of the pulse wave detection air bladder is connected to the pulse wave detection air bladder via the first tube. Furthermore, the third tube is connected to the sub air bladder. In addition to a pressure detector, the device can include a pulse detector and one or more wireless sensors.

The first, second, and third tubes, which are not visible to the user, are made of soft tubes. The third tube may be further connected to a damper device that increases the volume in proportion to the pressure and smooth the pressure. Other processes of pressurizing and depressurizing the cuff can be employed without departing from the scope of the disclosure.

The measurement device body is configurable to house the electronics which, for example, control the application of pressure to the cuff and analyze the resulting pressure data as well as transmitting the data (e.g. via Bluetooth) to another device. The measurement device acquires a measurement and then, for example, sends the acquired measurements, such as acquired blood pressure measurement, to a secondary device. The pump may be driven by a suitable power supply (e.g., battery) from a pump driver connected to a motor, and supplies the external air into the pump through an opening, thereby performing pressurization. A controller is provided which controls the operation of the device. Control of the operation is also modified in response to a detected cuff size. The air bladders of the cuff can then be pressurized by supplying the pressurizing air to a tube or bladder. A rapid exhaust valve/constant-rate exhaust valve can be provided that has a structure in which the opening area can be changed by the magnitude of the electromagnetic force in order to achieve a depressurization rate of 2 to 4 mmHg/sec.

A pressure sensor, such as a cuff pressure detector, is configurable to receive a compression pressure signal from the compression air bladder, in which the pulse wave component is attenuated via the fluid resistor, and the pressure change of the pulse wave detection air bladder. A pressure measuring unit for conversion into an analog electrical signal may be provided which is connected to the pressure sensor, and an A/D converter is connected to the pressure measuring unit. The A/D converter can then output a digital signal as a cuff pressure signal to a central controller.

The central controller is configurable to include a random access memory (RAM) for performing, for example, read and write of measurement data and analytical results, and a read only memory (ROM) for storing, as various control programs readable by the central controller, a pulse wave processor for detecting a pulse wave signal superposed on a cuff pressure signal, a cuff pressure controller for pressurizing and depressurizing the cuff (the compression air bladder, pulse wave detection air bladder, and sub air bladder), a blood pressure measuring unit for determining the blood pressure from the detected pulse wave change and compression cuff pressure signal. As would be evident to a person of skill in the art, the RAM can also function as a work area of programs to be processed by the central controller.

The central controller can also be in communication with a secondary electronic device such as a mobile device which can act as a blood pressure display for displaying the final blood pressure value, and to drivers for performing the above-mentioned driving control operations. The power supply from a power supply unit, including batteries, is performed such that the central controller can perform each operation required for blood pressure measurement by supplying power to the corresponding unit in accordance with the operation of a switch.

The blood pressure measurement device also has one or more of a power supply, a controller, a memory, a data i/o which is wired or wireless, a signal sensor, a pressure sensor, a controlled release valve, and a pump which is connected to the cuff by a connecting air pipe.

The blood pressure measurement device can have an on/off button and/or a start/stop button for taking a measurement. As will be appreciated the on/off button can include the functionality of start/stop. Alternatively, the device can power on when the cuff is unrolled from around the body. A display can be provided on the measurement device body. As will be appreciated by those skilled in the art, the measurement device body is illustrated in a rectangular form factor. However, other shapes can be employed without departing from the scope of the invention. For example, the measurement device body can be any suitable shape in two dimensions, such as square, round, oval, ovoid, etc. The measurement device body can also be any suitable shape in three dimension, such as cube, etc. Other shape variations will be apparent to those skilled in the art.

The distal end of the cuff can be secured by the use of a securement mechanism such as magnets or a hook and loop fastener such as Velcro® brand fasteners (available from Velcro Industries B.V. or similar fastening technologies available by other manufacturers) As will be appreciated by those skilled in the art, the cuff length vary depending on the characteristic of the subject being measured. Typically for adults, for example, the width of the cuff would be between 13 and 15 cm, and the length of the cuff would be sized to accommodate an upper-arm circumference between 22 cm and 37 cm—and thus would be longer than 22 cm-37 cm to provide additional length to allow the cuff to completely wrap-around the upper arm and then secure a portion of the cuff to itself. For example, a larger cuff could be sized to accommodate an upper arm circumference between 37 cm and 47 cm.

The cuff can be assembled from layers of textile and internal components. For example, an elongate layer which has a first flexible layer and a second flexible layer can be provided. Flexible layers can, for example, be fabric layers. A securement mechanism such as a hook and loop fastener (e.g., Velcro®) comprising a first hook fastener section and a second loop fastener section. Magnetic elements can be also provided which assist in securing the cuff in a closed position when not in use and achieving a low profile during storage and/or facilitate identification of an “on” or “off” state for the device. Semi-rigid mechanical fixing elements can also be provided. As will be understood by persons of skill in the art, blood pressure measurement devices include: a pump (that inflates the cuff located inside the device), a controlled release valve (that lets the air out at a controlled rate), a pressure gauge and a signal sensor (a microphone, really). The flexible layer can further include, for example a metal ring which allows the cuff to be looped around the upper arm of a patient during use. During deflation, when the signal sensor starts hearing the heartbeat, it means that blood pressure (“BP”) is less than or equal to the current air pressure in the cuff (systolic BP). When the signal sensors stop hearing the heartbeat, it means that blood pressure is greater than or equal to the current air pressure in the cuff (diastolic BP). This effectively means that the blood pressure is only sampled at each heartbeat. Slightly different algorithms can be applied as well, for example, sampling the BP during inflation (instead of during deflation). In this second case, when, during inflation the signal sensor starts hearing the heartbeat, it means that blood pressure is greater than or equal to the current air pressure in the cuff (diastolic BP). When the signal sensors stop hearing the heartbeat, it means that blood pressure is lower than or equal to the current air pressure in the cuff (systolic BP). This second method is faster, as it takes less time to inflate and deflate the cuff, but it is potentially less reliable due to the noise caused by the pump potentially interfering with the heart beat detection.

The systems and methods described herein rely on a variety of computer systems, networks, digital devices, and/or software apps for operation. In order to fully appreciate how the system operates an understanding of suitable computing systems is useful. The systems and methods disclosed herein are enabled as a result of application via a suitable computing system.

As will be appreciated by those skilled in the art, a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Some embodiments may be implemented in one or a combination of hardware, firmware and software. Embodiments may also be implemented as instructions stored on a non-transitory computer-readable storage medium, which may be read and executed by at least one processor to perform the operations described herein. A non-transitory computer-readable storage medium may include any mechanism for storing information in a form readable by a machine (e.g., a computer). For example, a non-transitory computer-readable storage medium may include read-only memory (ROM), random-access memory (RAM), magnetic disk storage media, optical storage media, flash-memory devices, and other non-transitory media.

In operation, the CPU fetches, decodes, and executes instructions, and transfers information to and from other resources via the computer's main data-transfer path, system bus. Such a system bus connects the components in the computing system and defines the medium for data exchange. Memory devices coupled to the system bus include random access memory (RAM) and read only memory (ROM). Such memories include circuitry that allows information to be stored and retrieved. The ROMs generally contain stored data that cannot be modified. Data stored in the RAM can be read or changed by CPU or other hardware devices. Access to the RAM and/or ROM may be controlled by memory controller. The memory controller may provide an address translation function that translates virtual addresses into physical addresses as instructions are executed.

In addition, the computing system can contain peripherals controller responsible for communicating instructions from the CPU to peripherals, such as, printer, keyboard, mouse, and data storage drive. Display, which is controlled by a display controller, is used to display visual output generated by the computing system. Such visual output may include text, graphics, animated graphics, and video. The display controller includes electronic components required to generate a video signal that is sent to display. Further, the computing system can contain network adaptor which may be used to connect the computing system to an external communications network.

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

The Open System Interconnection (OSI) model was established to standardize transmission between points over the Internet or other networks. The OSI model separates the communications processes between two points in a network into seven stacked layers, with each layer adding its own set of functions. Each device handles a message so that there is a downward flow through each layer at a sending end point and an upward flow through the layers at a receiving end point. The programming and/or hardware that provides the seven layers of function is typically a combination of device operating systems, application software, TCP/IP and/or other transport and network protocols, and other software and hardware.

Wireless networks can incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

Computing system, described above, can be deployed as part of a computer network used to achieve the desired technical effect and transformation. In general, the above description for computing environments applies to both server computers and client computers deployed in a network environment. An exemplary illustrative networked computing environment, with a server in communication with client computers via a communications network. A server may be interconnected via a communications network (which may be either of, or a combination of a fixed-wire or wireless LAN, WAN, intranet, extranet, peer-to-peer network, virtual private network, the Internet, or other communications network) with a number of client computing environments such as tablet personal computer, mobile telephone, smart phone, personal computer, and personal digital assistant. In a network environment in which the communications network is the Internet, for example, server can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment can utilize various data security protocols such as secured socket layer (SSL) or pretty good privacy (PGP). Each client computing environment can be equipped with operating system operable to support one or more computing applications, such as a web browser (not shown), or other graphical user interface (not shown), or a mobile desktop environment (not shown) to gain access to server computing environment.

In operation, a user (not shown) may interact with a computing application running on a client computing environment to obtain desired data and/or computing applications. The data and/or computing applications may be stored on server computing environment and communicated to cooperating users through client computing environments over exemplary communications network. The computing applications, described in more detail below, are used to achieve the desired technical effect and transformation set forth. A participating user may request access to specific data and applications housed in whole or in part on server computing environment. These data may be communicated between client computing environments and server computing environments for processing and storage. Server computing environment may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### EXAMPLES

Example 1: A user opens the blood pressure measurement device from its storage configuration by unwrapping the cuff from a storage position around the device housing. The blood pressure measurement device is secured to the user's arm by wrapping the cuff around the arm and positioning the sensor to optimally detect blood pressure. The blood pressure measurement device is then activated to begin detecting the user's blood pressure. Concurrently, or substantially concurrently, a display (device display, computer display, mobile device display, etc.) presents input (visual, audio, or both) to the user during the process of obtaining the blood pressure measurement. At the conclusion of obtaining the blood pressure measurements, the results may be either one or both of presented to the user (by visual, audio or both) and presented to a third party (doctor, health buddy, etc.).

Example 2: A user opens the blood pressure measurement device from its storage configuration. Activating the pulley powers by rotating the pulley away from the blood pressure measurement device places the device into a ready configuration. Thereafter the user secures the blood pressure measurement device to his or her arm such that the blood pressure sensor is positioned to optimally detect blood pressure. The blood pressure measurement device is activated by a command provided by the blood pressure measurement device or a computing system connected wirelessly to the blood pressure measurement device. Such command may be user triggered (e.g. by pressing a button on a touch screen, or activating any other user interface element) or generated automatically by the system to begin detecting the user's blood pressure. Concurrently, or substantially concurrently, a display (blood pressure measurement device display, computer display, mobile device display, etc.) presents input (visual, audio, or both) to the user during the process of obtaining the blood pressure measurement. At the conclusion of obtaining the blood pressure measurement, the results may be either one or both of presented to the user (by visual, audio or both) and presented to a third party (doctor, health buddy, etc.).

Example 3: The user selects a blood pressure cuff that is sized to fit the user. The user then secures the blood pressure cuff to the body of the blood pressure measurement device before wrapping the cuff around the user's arm. The blood pressure measurement device determines the presence and size of the blood pressure cuff attached to the blood pressure measurement device and then selects an algorithm for operation of the blood pressure measurement device that corresponds to the algorithm assigned to the size of the blood pressure cuff. The blood pressure measurement device is activated by a command provided by the blood pressure measurement device or a computing system connected wirelessly to the blood pressure measurement device. Such command may be user triggered (e.g. by pressing a button on a touch screen, or activating any other user interface element) or generated automatically by the system to begin detecting the user's blood pressure. Concurrently, or substantially concurrently, a display (blood pressure measurement device display, computer display, mobile device display, etc.) presents input (visual, audio, or both) to the user during the process of obtaining the blood pressure measurement. At the conclusion of obtaining the blood pressure measurement, the results may be either one or both of presented to the user (by visual, audio or both) and presented to a third party (doctor, health buddy, etc.).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that any claims presented define the scope of the invention and that methods and structures within the scope of the claims and their equivalents are covered thereby.

What is claimed:

1. A blood pressure measurement device comprising:
a cuff for wrapping around a limb of a subject, the cuff further having an inflatable bladder formed integrally therein, wherein the cuff is substantially rectangular in shape having a first surface, a second surface, a first end, a second end, and two sides, one or more tubes positioned within the cuff in communication with the inflatable bladder, an exhaust valve, a cuff securement mechanism, and a housing mating base secured to a surface of the cuff with a vertical base surface extending from a planar base surface;
a housing for the blood pressure measurement device wherein the housing is removably securable to the housing mating base of the cuff and further wherein the housing includes a dock and a button, and one or more of a controller, a memory, a pump, a power supply, and a motor; and
wherein the vertical base surface of the housing mating base interfaces with the dock and button of the housing to identify a presence and size of the cuff.

2. The blood pressure measurement device of claim 1 further comprising one or more of an on-off button and a start-stop button.

3. The blood pressure measurement device of claim 1 wherein the blood pressure measurement device is in wireless communication with one or more displays.

4. The blood pressure measurement device of claim 1 further comprising a pulley.

5. The blood pressure measurement device of claim 4 wherein the pulley engages the housing along an end and further wherein movement of the pulley away from the housing activates the blood pressure measurement device from an off condition to an on condition.

6. The blood pressure measurement device of claim 1 further comprising one or more of a pressure detector, a pulse detector and one or more wireless sensors.

7. The blood pressure measurement device of claim 1 wherein the cuff securement mechanism is at least one of magnets and a hook and loop fastener.

8. The blood pressure measurement device of claim 1 wherein the blood pressure measurement device is in wireless communication with one or more displays.

9. The blood pressure measurement device of claim 1 wherein an operation of the blood pressure measurement device changes in response to an identified size of the cuff.

10. The blood pressure measurement device of claim 1 wherein a surface of the housing mating base has a raised structure sized to receive a recess on an exterior surface of the housing.

11. The blood pressure measurement device of claim 1 further comprising a transmitter for wirelessly transmitting blood pressure data to a second device.

12. A method for measuring blood pressure using a blood pressure measurement device comprising:
selecting a cuff;
securing the cuff to a housing if the blood pressure measurement device;
selecting an algorithm for operating the blood pressure measurement device based on a size of the selected cuff;
wrapping the cuff around a limb of a subject the cuff having an inflatable bladder formed integrally therein, wherein the cuff is substantially rectangular in shape having a first surface, a second surface, a first end, a second end, and two sides, and a securement mechanism, one or more tubes positioned within the cuff in communication with the inflatable bladder, an exhaust valve; the housing includes a dock and a button, and one or more of a controller, a memory, a pump, a power supply, and a motor; and
delivering an output of a measured blood pressure.

13. The method for measuring blood pressure of claim 12 further comprising the step of turning the blood pressure measurement device on.

14. The method for measuring blood pressure of claim 12 further comprising measuring one or more of a blood pressure and a pulse.

15. The method for measuring blood pressure of claim 12 further comprising delivering the output of the blood pressure measurement device wirelessly a second device.

16. The method for measuring blood pressure of claim 15 further comprising providing an acquired blood pressure measurement to a third party device that is different than the second device.

17. The method for measuring blood pressure of claim 16 wherein the step of providing an acquired blood pressure measurement to a third party device occurs one or more of automatically and wirelessly.
